(19) United States
(12) Patent Application Publication
(10) Pub. No.: US 2018/0000422 A1
(43) Pub. Date: Jan. 4, 2018

(54) BIOLOGICAL INFORMATION DETECTION DEVICE AND CHAIR INCLUDING BIOLOGICAL INFORMATION DETECTION DEVICE

(71) Applicant: Shigetomo Mitani, Aichi (JP)

(72) Inventor: Shigetomo Mitani, Aichi (JP)

(73) Assignee: OMRON AUTOMOTIVE ELECTRONICS CO., LTD., Aichi (JP)

(21) Appl. No.: 15/641,123

(22) Filed: Jul. 3, 2017

(30) Foreign Application Priority Data
Jul. 4, 2016 (JP) 2016-132701

(51) Int. Cl. A61B 5/00; A61B 5/0205; A61B 5/05; A61B 5/024; A61B 5/08

(52) U.S. Cl. CPC A61B 5/6893; A61B 5/0205; A61B 5/0816; A61B 5/024; A61B 2503/22; A61B 5/05

(57) ABSTRACT

A biological information detection device is installed in equipment including a body support portion, and detects biological information of a user of the equipment. The device includes: a detection unit including: a transmitter which transmits a radio wave toward a part of the body supported by the body support portion; and a receiver which receives a reflected wave obtained as the radio wave transmitted by the transmitter and then reflected on a part of the body; and a detection support portion which forms a part of the body support portion, and which supports a part of the body to which the transmitter transmits the radio wave. Hardness with respect to a part of the body at the detection support portion is softer than hardness with respect to a part of the body at the body support portion excluding the detection support portion.

### CROSS-REFERENCES TO RELATED APPLICATIONS

This application is based upon and claims the benefit of priority from Japanese Patent Application No. 2016-132701, filed on Jul. 4, 2016; the entire contents of which are incorporated herein by reference.

### FIELD

One or more embodiments of the invention relate to a biological information detection device and a chair including a biological information detection device, and particularly relate to a biological information detection device which acquires biological information by supporting a living body, and a chair including the biological information detection device.

### BACKGROUND

In the related art, a technique is known which supports a living body and acquires biological information such as a heart rate and a respiratory rate of a living body. For example, JP-A-2001-260698 discloses a heartbeat respiration detection device which aims to accurately detect the heart rate and the respiratory rate, even in a case where an entire body of a driver moves back and forth due to acceleration or deceleration. This heartbeat respiration detection device includes a transceiver which transmits an electromagnetic wave to the driver and receives a reflected wave thereof, and a calculation processing unit which obtains the heart rate and the respiratory rate by performing calculations based on the frequency change between the transmitted wave and the reflected wave. The transceiver is attached to a surface on which a seat belt comes into contact with the body of the driver. In this manner, even if the body of the driver moves back and forth during driving, the transceiver is always in close contact with the driver. Therefore, the heart rate and the respiratory rate can be accurately detected.

JP-A-2002-045350 discloses a biological signal detection device which aims to detect an accurate biological signal over a long period of time without any mechanical time-dependent change in a configuration member even in a case of a long-term use. The biological signal detection device includes a box body having at least a top plate and a magnetic sensor disposed inside the box body. The top plate is configured to include a material having flexibility and soft magnetism. JP-A-2002-045350 also discloses the biological signal detection device configured to include a rectangular fixing plate, a spacer member placed on the fixing plate, a magnetic sensor disposed inside a hole formed in the spacer member, and a seat-shaped soft magnetic member placed on the spacer member. The fixing plate is made of a nonmagnetic material such as aluminum or a synthetic resin. The spacer member is made of a nonmagnetic material such as polystyrene foam or urethane foam which is a foaming agent. The hole for accommodating the magnetic sensor is formed at a substantially center position of the spacer member. The height of the magnetic sensor is lower than that of the spacer member. In a state where the soft magnetic member is placed on the spacer member, a gap is formed between the soft magnetic member and the spacer member. For example, the biological signal detection device is installed under a bed on which a measurement target person takes a rest, under a pillow, or on a toilet seat. Then, a distance between the magnetic sensor and the soft magnetic member is changed due to fine movements of the measurement target person, thereby causing the magnetic sensor to output a fine movement signal corresponding to the change in the distance.

JP-A-2009-055997 discloses a biological vibration frequency detection device which is excellent in detection accuracy. This biological vibration frequency detection device includes a plurality of microwave transceivers which transmit microwaves to a subject and receive reflected waves thereof, and a CPU which performs cross correlation processing using a heartbeat signal based on a plurality of the reflected waves and which detects a heart rate of the subject using a processing result thereof. In this manner, the heart rate can be detected using a result of the cross-correlation processing. Therefore, even if one of the reflected waves contains noise, it is possible to minimize the influence of this noise, and the detection accuracy of the heart rate can be improved.

JP-A-2010-120493 discloses a biological signal detection device which prevents poor accuracy in a biological signal of an occupant. The biological signal detection device detects the biological signal of the occupant of a vehicle, and includes a sensor unit which causes a radio wave type non-modulated Doppler sensor to detect a movement of the occupant, a biological signal extraction unit which extracts the biological signal of the occupant, based on a phase change in an output of the sensor unit, a distance calculation unit which calculates an estimated distance between the sensor unit and the occupant, based on an integrated value of a phase change amount of the output of the sensor unit, and a biological signal output determination unit which determines reliability of the biological signal, based on the estimated distance and which stops the output of the biological signal in a case where the reliability is low. In the biological signal detection device, the sensor unit is installed behind the occupant such as a backrest portion of a seat on which the occupant sits. In a case where the estimated distance is equal to or greater than a predetermined distance threshold, the biological signal output determination unit determines that the reliability of the biological signal is low, and stops the output of the biological signal.

JP-A-2014-087473 discloses a biological information detection device which can reliably detect biological information by properly providing a living body detection sensor in relation to a configuration of an elastic member for supporting a living body. This biological information detection device detects the biological information by disposing an elongated living body detection sensor in the elastic member for supporting the living body and by bringing the living body into pressing contact with the living body detection sensor. In the elastic member, a portion for supporting an intermediate portion of the living body detection sensor is more flexible than a portion for supporting both end portions. For example, as the elastic member, a seat base material used for the portion for supporting the intermediate portion is formed of a material which is more flexible than that of the seat base material used for the portion for supporting both end portions. Alternatively, the elastic member has a void (recess or cutout) on a rear surface side of the portion for supporting the intermediate portion of the living body detection sensor. In this biological information detection device, if the occupant sits on a seat cushion and comes into pressing contact with a seat backrest, the recess and cutout of the elastic member are more likely to be bent than both end portions. Accordingly, the void is bent together with the living body detection sensor. As the occupant breathes, pressure fluctuations (vibrations) occur in a piezoelectric body in the intermediate portion of the living body detection sensor, thereby outputting each voltage signal generated by a piezoelectric effect.

JP-A-2015-123359 discloses a vehicle seat which has a seat-like sensor installed to detect an electric signal corresponding to a biological potential of a sitting person. The vehicle seat can stably measure a biological electric signal such as an electrocardiographic signal of the sitting person by removing noise generated by static electricity. A seat backrest of the vehicle seat includes a seat-like sensor which can measure a heart rate of the sitting person. A cushion pad is placed on a seat frame, and the seat backrest is covered with a trim cover. A band-shaped conductive cloth extending in a vertical direction and having a width of approximately 20 mm is arrayed on an outer surface of the trim cover. A portion having the conductive cloth arrayed thereon has a three-layered structure including the conductive cloth, a skin, and a wadding material from the outside. A free end of the conductive cloth drawn into the seat backrest is provided with a J-hook, and is latched by a lower frame installation portion. In this manner, a portion of the conductive cloth and the lower frame installation portion are brought into contact with each other so that both of these are electrically connected to each other.

### SUMMARY

One or more embodiments of the invention provide a biological information detection device and a chair including a biological information detection device which support a living body and accurately detects biological information such as a heart rate or a respiratory rate of the living body.

According to one or more embodiments of the invention, there is provided a biological information detection device which is installed in equipment including a body support portion which supports a body, and which detects biological information of a user of the equipment. The biological information detection device includes: a detection unit including: a transmitter which transmits a radio wave toward a part of the body supported by the body support portion; and a receiver which receives a reflected wave obtained as the radio wave transmitted by the transmitter and then reflected on a portion of the body; and a detection support portion which forms a part of the body support portion, and which supports a part of the body to which the transmitter transmits the radio wave. Hardness with respect to a part of the body at the detection support portion is softer than hardness with respect to a part of the body at the body support portion excluding the detection support portion.

According to this configuration, the body support portion excluding the detection support portion is relatively harder so as to minimize distance variations between a surface of a living body and the detection unit, and the detection support portion is relative softer so as to minimize surface compression of the living body. In this manner, it is possible to provide the biological information detection device which supports the living body and accurately acquires the biological information such as a heart rate and a respiratory rate of the living body.

In the aspect, the detection support portion may include an elastic member.

According to this configuration, since the elastic member absorbs vibrations generated from the outside, the biological information can be accurately acquired.

In the aspect, the biological information detection device may further include a frame body including: a bottom portion on which the detection unit is disposed; and a side portion which is erected from the bottom portion to a surface on which the detection support portion supports a part of the body. The detection support portion may be disposed across an inside of the side portion.

According to this configuration, since a distance between a portion of the body and the detection unit is maintained constant, the biological information can be accurately acquired.

According to one or more embodiments of the invention, there is provided a chair including the above-described biological information detection device, a support body that directly or indirectly supports the detection unit, and a seat surface including the body support portion.

According to this configuration, it is possible to provide the chair which supports the living body and accurately acquires the biological information such as the heart rate and the respiratory rate of the living body.

According to one or more embodiments of the invention, it is possible to provide the biological information detection device and the chair including the biological information detection device which support the living body and accurately acquire the biological information such as the heart rate and the respiratory rate of the living body.

### BRIEF DESCRIPTION OF THE DRAWINGS

FIG. 1A is a schematic view illustrating an example in which a biological information detection device according to a first embodiment of the invention is installed in a vehicle seat;

FIG. 1B is an enlarged sectional view of the biological information detection device installed on a seat surface;

FIG. 2A is a plan view of the biological information detection device according to the first embodiment of the invention;

FIG. 2B is a sectional view thereof;

FIG. 3A is a plan view of a biological information detection device according to a second embodiment of the invention;

FIG. 3B is a sectional view thereof; and

FIG. 4 is a schematic view illustrating an example in which the biological information detection device according to the second embodiment of the invention is installed in a vehicle seat.

### DETAILED DESCRIPTION

In embodiments of the invention, numerous specific details are set forth in order to provide a thorough understanding of the invention. However, it will be apparent to one of ordinary skill in the art that the invention may be practiced without these specific details. In other instances, well-known features are not described in detail to avoid obscuring the invention.

### First Embodiment

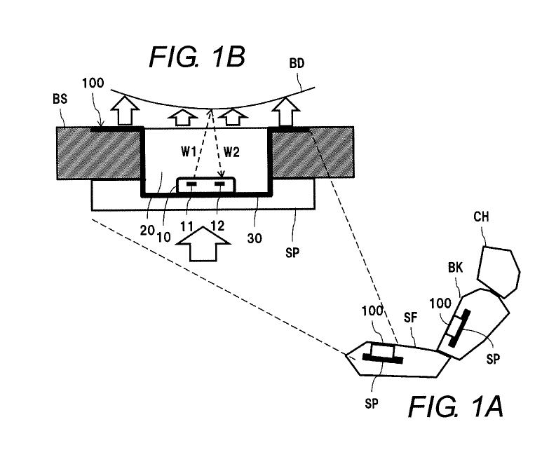

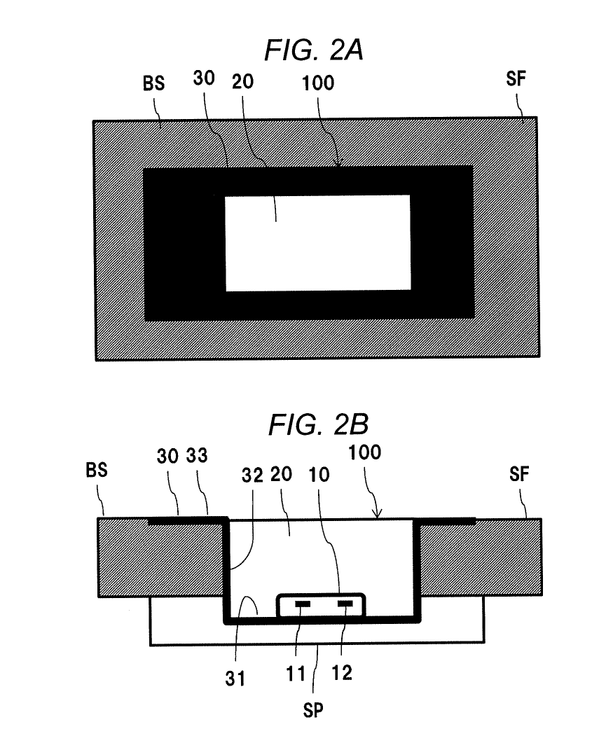

A biological information detection device 100 according to a first embodiment will be described with reference to FIGS. 1A to 2B. The biological information detection device 100 is installed in equipment CH having a body support portion BS for directly or indirectly supporting a portion of a human body BD so as to detect biological information of a user of the equipment CH. Here, the equipment (general term of tools, instruments, and machines) having the body support portion BS for supporting the portion of the human body BD specifically means, for example, a chair or a sofa on which a person sits, a bed on which a person lies, body inspection equipment installed in a hospital, and a seat installed in a vehicle or an aircraft so that a person sits thereon.

The body support portion BS for supporting the portion of the human body BD indicates a seat surface and a backrest surface of the chair, and a mattress upper surface of the bed. The body support portion BS may be directly or indirectly brought into contact with the portion of the human body BD. Alternatively, a person who wears clothes may be supported while indirectly being in contact with the body support portion BS. A part of the body BD generally indicates a buttock or a thigh on the seat surface of the chair, and a back on a backrest of the chair or the bed. In the body inspection equipment, the portion may indicate any limb of a person.

In the embodiments described herein, the biological information of a user indicates a heart rate (pulse rate), a magnitude of a pulse wave, a respiratory rate and a magnitude of respiration. The biological information does not include coughing or sneezing which causes a movement of skin or a muscle not derived from the above-described biological information.

As illustrated in FIG. 1A, the biological information detection device 100 in the embodiment described herein is installed in a so-called seat CH serving as equipment mounted on a vehicle, that is, on a seat surface SF or a backrest portion BK of the seat CH. The seat surface SF or a surface of the backrest portion BK is covered with skin, and a surface on which a part of the body BD is in contact with the body support portion BS is the skin. In this case, the body support portion BS is an elastic member, a so-called cushion, which is disposed on the seat surface SF or the backrest portion BK, and supports a portion of the weight of the body BD. The part of the body BD indicates a thigh or a back of a user on a seat CH, who is a driver of the vehicle, for example. The seat CH internally includes a support body SP which is a strong frame, and the biological information detection device 100 is attached to the support body SP.

As illustrated in FIGS. 1B, 2A, and 2B, the biological information detection device 100 includes a detection unit 10 that detects the biological information, a detection support portion 20 that configures a part of the body support portion BS and supports a part of the body BD, and a frame body 30 on which the detection unit 10 is mounted. The detection unit 10 has a transmitter 11 that transmits a radio wave (transmitted wave W1) toward the part of the body BD supported by the body support portion BS, and a receiver 12 that receives a reflected wave W2 in such a way that the radio wave transmitted by the transmitter 11 is reflected on the part of the body BD. The radio wave (transmitted wave W1) to be transmitted may employ any frequency as long as the frequency allows the radio wave to be reflected on the skin surface of a living body. However, it is normally preferable to employ a microwave. FIG. 1B is an enlarged sectional view of the biological information detection device 100 disposed on the seat surface SF of the seat CH illustrated in FIG. 1A.

The reflected wave W2 received by the receiver 12 is processed in order to detect the biological information such as normal heartbeats and breathing, and is transmitted to an external device using the biological information. Since the skin surface of the living body finely moves in response to the heartbeats and breathing, the detection unit 10 detects its movement, based on a change in the frequency of the transmitted wave W1 and the reflected wave W2. Therefore, in order not to limit the fine movement of the surface of the living body, it is preferable that a force such as compression is not applied to the part of the body BD for detecting the biological information.

Similarly to the body support portion BS, the detection support portion 20 supports the part of the body BD, and supports the part of the body BD to which the radio wave is transmitted by the transmitter 11. That is, the detection support portion 20 is a portion that supports the part of the body BD for acquiring the biological information of the user of the seat CH. The detection support portion 20 supports the part of the body BD which reflects the radio wave transmitted by the transmitter 11 as the reflected wave W2. Hardness with respect to the part of the body BD at the detection support portion 20 is softer than hardness with respect to the part of the body BD at the body support portion BS excluding the detection support portion 20.

The hardness is so-called rigidity, which may be the hardness in a hardness test (JIS K 6401) for flexible polyurethane foam used for a chair or a mattress, for example. The hardness is indicated in Newton, and those having a smaller value are softer than those having a greater value. Therefore, the hardness value of the detection support portion 20 is smaller than the hardness value of the body support portion BS excluding the detection support portion 20.

As illustrated in FIG. 1B, the hardness with respect to the part of the body BD is indicated by the size of the arrow. The body support portion BS excluding the detection support portion 20 is relatively harder, and supports the part of the body BD with a strong force. On the other hand, the detection support portion 20 is relatively softer, and supports the part of the body BD with a weak force. In this way, since the body support portion BS excluding the detection support portion 20 is relatively harder, this portion supports the majority of the body weight of the body BD. In this manner, it is possible to minimize the distance variations between the surface of the living body and the detection unit 10.

Since the detection support portion 20 is relatively softer, it is possible to minimize the surface compression of the living body. In this way, the distance variations are minimized between the surface of the living body and the detection unit 10 by the body support portion BS excluding the detection support portion 20, and the surface compression of the living body is minimized by the detection support portion 20. Accordingly, it is possible to provide the biological information detection device 100 which supports the living body and accurately acquires the biological information such as the heart rate and the respiratory rate of the living body.

The hardness with respect to the part of the body BD may be achieved based on the hardness of the material itself, which is the elastic member used for the detection support portion 20, or may be realized based on the structure such as the size and the shape. For example, in the detection support portion 20 and the body support portion BS excluding the detection support portion 20, the elastic member (for example, flexible polyurethane foam) having the same hardness is used. However, the elastic member of the detection support portion 20 is thinned, or a cutout is disposed therein. Thus, the elastic member is likely to be bent. In this manner, the detection support portion 20 in contact with the part of the body BD may be relatively softer. Since the detection support portion 20 is configured to include the elastic member, the elastic member can absorb vibrations generated from the outside, and the biological information can be accurately acquired.

The frame body 30 has a bottom portion 31 in which the detection unit 10 is disposed, a side portion 32 which is erected from the bottom portion 31 to a surface on which the detection support portion 20 supports the part of the body BD, and a flange portion 33 which extends outward in an upper end of the side portion 32. A lower surface of the bottom portion 31 is attached to the strong support body SP included in the seat CH. The frame body 30 itself is attached to the seat CH. The bottom portion 31 has a rectangular shape in a plan view, and the detection unit 10 is fixedly disposed on the upper surface of the rectangular bottom portion 31. The side portion 32 is erected from the edge of the bottom portion 31 to the surface on which the detection support portion 20 supports the part of the body BD. Accordingly, the detection unit 10 is disposed so as to be accommodated in the frame body 30, and is indirectly supported by the support body SP.

The size of the bottom portion 31 and the height of the side portion 32 may have a relationship to such an extent that even if the radio wave transmitted by the transmitter 11 of the detection unit 10 spreads, the radio wave directly comes into contact with the part of the body BD. The height of the side portion 32 is set to the height which coincides with the upper surface of the body support portion BS. The flange portion 33 spreads outward in a shape substantially similar to that of the bottom portion 31 in the upper end of the side portion 32, and is configured to coincide with the upper surface of the body support portion BS.

In this case, the detection support portion 20 is disposed over the entire surface inside the side portion 32 in a plan view, and from the bottom portion 31 up to the surface on which the detection support portion 20 supports the part of the body BD. It is preferable that the frame body 30 is formed of metal or a resin which is harder than the body support portion BS or the detection support portion 20. Since the frame body 30 is provided, the distance between the part of the body BD and the detection unit 10 is maintained constant. Accordingly, it is possible to accurately detect the biological information such as the heartbeats or breathing by reducing the influence of displacement of the surface of the living body which is derived from a factor other than the heartbeat or breathing, such as vibrations of the vehicle.

### Second Embodiment

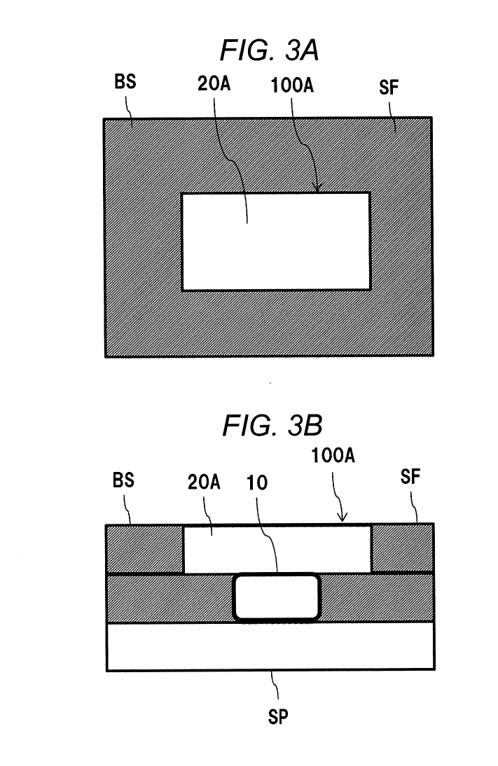

Referring to FIGS. 3A and 3B, a biological information detection device 100A according to a second embodiment will be described. In order to avoid redundant description, the same reference numerals will be given to the same elements as those of the above-described embodiment, and different elements will be mainly described. The biological information detection device 100A includes the detection unit 10 which detects the biological information and a detection support portion 20A which configures a part of the body support portion BS and supports a part of the body BD. The biological information detection device 100A does not have the frame body 30 which is present in the above-described embodiment.

The body support portion BS includes two layers. A hole for accommodating the detection unit 10 is disposed on a lower layer close to the support body SP of the two layers. A hole for accommodating the body support portion BS is disposed on an upper layer which is in contact with the part of the body BD of the two layers. The detection unit 10 is directly attached to the support body SP, and is disposed in the hole disposed on the lower layer of the body support portion BS. The detection support portion 20A formed with approximately the same thickness as that of the upper layer of the body support portion BS is disposed in the hole disposed on the upper layer of the body support portion BS, and is configured to coincide with the body support portion BS on the seat surface SF.

The size in a plan view of the hole disposed on the upper layer which is in contact with the part of the body BD is larger than the size in a plan view of the hole disposed on the lower layer closer to the support body SP. The size in a plan view of the hole disposed in the upper layer which is in contact with the part of the body BD and the thickness of the upper layer may have a relationship to such an extent that even if the radio wave transmitted by the transmitter 11 of the detection unit 10 spreads, the radio wave directly comes into contact with the part of the body BD.

The body support portion BS excluding the detection support portion 20A is relatively harder, and supports the part of the body BD with a strong force. On the other hand, the detection support portion 20A is relatively softer, and supports the part of the body BD with a weak force. In this way, since the body support portion BS excluding the detection support portion 20A is relatively harder, this portion supports the majority of the body weight of the body BD. In this manner, it is possible to minimize the distance variations between the surface of the living body and the detection unit 10. Since the detection support portion 20A is relatively softer, it is possible to minimize the surface compression of the living body. In this way, the distance variations are minimized between the surface of the living body and the detection unit 10 by the body support portion BS excluding the detection support portion 20A, and the surface compression of the living body is minimized by the detection support portion 20A. Accordingly, it is possible to provide the biological information detection device 100A which supports the living body and accurately acquires the biological information such as the heart rate and the respiratory rate of the living body.

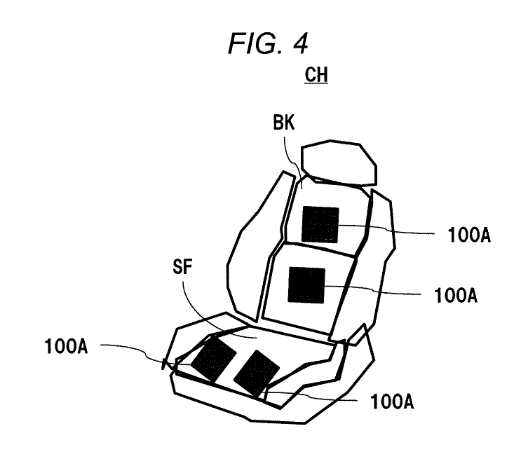

As illustrated in FIG. 4, the biological information detection device 100A can be disposed in various locations of the seat CH. In a case of acquiring pulsation, it is preferable that the biological information detection device 100A is disposed in the upper portion of the backrest portion BK, close to the location of the heart. In a case of acquiring the biological information which does not receive the influence of a steering operation, it is preferable that the biological information detection device 100A is disposed particularly around the waist along the spine axis. In a case of acquiring the biological information least affected by the movement of the upper body such as breathing, it is preferable that the biological information detection device 100A is disposed on the seat surface SF close to the thigh. In this way, the biological information detection device 100A is incorporated in the body support portion BS of the seat CH. Accordingly, it is possible to avoid the influence of seat components, and it is possible to easily dispose the biological information detection device 100A in a location where the biological information can be easily acquired. The seat CH (chair CH) including the biological information detection device 100A can support the living body, and can accurately acquire the biological information such as the heart rate and the respiratory rate of the living body.

While the invention has been described with respect to a limited number of embodiments, those skilled in the art, having benefit of this disclosure, will appreciate that other embodiments can be devised which do not depart from the scope of the invention as disclosed herein. Accordingly, the scope of the invention should be limited only by the attached claims.

1. A biological information detection device which is installed in equipment comprising a body support portion which supports a body, and which detects biological information of a user of the equipment, the biological information detection device comprising:
a detection unit comprising: a transmitter which transmits a radio wave toward a part of the body supported by the body support portion; and a receiver which receives a reflected wave obtained as the radio wave transmitted by the transmitter and then reflected on a part of the body; and
a detection support portion which forms a part of the body support portion, and which supports a part of the body to which the transmitter transmits the radio wave,
wherein hardness with respect to a part of the body at the detection support portion is softer than hardness with respect to a part of the body at the body support portion excluding the detection support portion.

2. The biological information detection device according to claim 1,
wherein the detection support portion comprises an elastic member.

3. The biological information detection device according to claim 1, further comprising:
a frame body comprising: a bottom portion on which the detection unit is disposed; and a side portion which is erected from the bottom portion to a surface on which the detection support portion supports a part of the body,
wherein the detection support portion is disposed across an inside of the side portion.

4. A chair comprising:
the biological information detection device according to claim 1;
a support body that directly or indirectly supports the detection unit; and
a seat surface comprising the body support portion.
